(19) United States
(12) Patent Application Publication
(10) Pub. No.: US 2005/0196868 A1
(43) Pub. Date: Sep. 8, 2005

(54) Use of free hemoglobin and its surrogate markers to detect and monitor pulmonary hypertension

(75) Inventors: Jeffrey A. Kline, Charlotte, NC (US); John Zagorski, Charlotte, NC (US)

Correspondence Address: KENNEDY COVINGTON LOBDELL & HICKMAN, LLP, 214 N. TRYON STREET, HEARST TOWER, 47TH FLOOR, CHARLOTTE, NC, 28202, US

(73) Assignee: The Charlotte-Mecklenburg Hospital Authority, Charlotte, NC (US)

(21) Appl. No.: 11/071,745

(22) Filed: Mar. 3, 2005

Related U.S. Application Data
(60) Provisional application No. 60/549,629, filed on Mar. 3, 2004.

(51) Int. Cl. G01N 33/72

(52) U.S. Cl. 436/66

(57) ABSTRACT

A method for diagnosing and monitoring pulmonary hypertension using free hemoglobin, as well as surrogates for free hemoglobin, as markers for pulmonary hypertension. Bodily fluids, such as blood, serum, plasma, urine and/or breathe condensate may be collected and analyzed to determine the concentration of free hemoglobin or surrogates of free hemoglobin. The concentration indicates the presence or absence of pulmonary hypertension.

### CROSS-REFERENCE TO RELATED APPLICATIONS

This application is entitled to the benefit of, and claims priority to provisional U.S. Patent Application Ser. No. 60/549,629, filed on Mar. 3, 2004, which is incorporated herein by reference in its entirety.

### BACKGROUND OF THE PRESENT INVENTION

1. Field of the Present Invention

The present invention relates generally to diagnosing and monitoring pulmonary hypertension, and, in particular, to the use of free hemoglobin, as well as surrogates for free hemoglobin, as markers for detecting and monitoring pulmonary hypertension.

2. Background

Pulmonary hypertension contributes to the severity and progression of several disease states, including thrombotic embolism to the lung, sickle cell disease, acute fat embolism syndrome, tumor embolism syndrome, pulmonary schistomasiasis, right-sided congestive heart failure and primary pulmonary hypertension. Patients with any of these conditions complicated by pulmonary hypertension are far more likely to be disabled or die compared with patients who do not have pulmonary hypertension.

Several treatments aimed at reducing pulmonary hypertension once it is diagnosed are available; however, current methods of detecting and monitoring pulmonary hypertension have significant disadvantages. One such diagnostic method includes inserting a pulmonary arterial catheter through a large vein in the neck or chest. Potential adverse side effects of this procedure include sustaining a punctured lung, incurring damage to blood vessels or the heart or experiencing an abnormal heart rhythm. This procedure requires expensive equipment and special competence to perform and interpret the results.

Another method, transthoracic or transesophageal Doppler-echocardiography, represents a non-invasive option to pulmonary artery catheterization. The Doppler method measures the velocity of blood flow that travels in the reverse direction across the tricuspid valve of the heart. This method has the drawbacks of requiring specialized Doppler equipment to perform the measurement, a skilled operator to acquire the data, and a specialist physician to interpret the results. An additional disadvantage is that the mathematical equation used to predict the pulmonary arterial systolic pressure requires the value of the right atrial pressure to be assumed, thus introducing uncertainty into the analysis.

Accordingly, it would be advantageous to have a noninvasive test or set of tests to detect and monitor the presence of pulmonary hypertension.

### SUMMARY OF THE PRESENT INVENTION

The present invention comprises various methods of diagnosing, detecting, assessing and monitoring pulmonary hypertension and various other conditions in mammalian subjects using at least one of haptoglobin, prehaptoglobin, free hemoglobin, biliverdin, bilirubin, nitric oxide, carbon monoxide or the ratio of carbon dioxide to oxygen as a marker.

Broadly defined, the present invention according to one aspect is a method of detecting and monitoring the presence of acute or chronic pulmonary hypertension, including collecting a bodily fluid and measuring the concentration of haptoglobin or its precursor, prehaptoglobin, in the bodily fluid.

The present invention, according to another aspect of the invention is a method of detecting and monitoring the presence of acute or chronic pulmonary hypertension, including collecting a bodily fluid and measuring the concentration of free hemoglobin in the bodily fluid.

The present invention, according to another aspect of the invention is a method of detecting and monitoring the presence of acute or chronic pulmonary hypertension, including collecting a bodily fluid and measuring the concentration of a product of heme catabolism in the bodily fluid.

In features of this aspect, the product of heme catabolism may be biliverdin or bilirubin.

The present invention, according to another aspect of the invention is a method of detecting and monitoring the presence of acute or chronic pulmonary hypertension, including collecting expired breath from a patient and measuring the concentration of nitric oxide and carbon monoxide in the expired breath.

The present invention, according to another aspect of the invention is a method of detecting and monitoring the presence of acute or chronic pulmonary hypertension, including collecting expired breath from a patient and measuring the concentration of carbon dioxide to oxygen in the expired breath.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawing, wherein:

FIG. 1 is a graphical representation of a relationship between pulmonary hypertension and free hemoglobin and its surrogate markers.

FIG. 2 is a graphical representation of the results of the ELISA assay analysis of Example 2.

FIG. 3 is a graphical representation of the partial pressure of CO₂ versus partial pressure of O₂ for 178 patients using the mean values of the first two deep exhaled breaths in Example 3.

FIG. 4 is a graphical representation of the partial pressure of CO₂ versus partial pressure of O₂ for 178 patients using the mean end-tidal partial pressure of CO₂ and partial pressure of O₂ obtained during the first minute of tidal breathing in Example 3.

### DETAILED DESCRIPTION OF THE PREFERRED EMBODIMENTS

The following description of the preferred embodiment(s) is merely exemplary in nature and is in no way intended to limit the invention, its application, or uses.

Pulmonary hypertension may be defined as a condition of increased mean pulmonary artery pressure over that which is normal and healthy for a particular person. Specifically, it may be defined as a mean pulmonary artery pressure greater than 25 mm Hg at rest or greater than 30 mm Hg during exercise, with increased pulmonary vascular resistance. However, for some, pulmonary hypertension, and more particularly the adverse effects thereof, may not be experienced until they have a mean pulmonary artery pressure greater than 40 mm Hg or higher at rest. The diagnosis should be made with a clinical assessment of multiple factors including, but not limited to, hemodynamic parameters, medical history and histological findings.

Pulmonary hypertension may be generally categorized as either precapillary, with changes limited to the arterial side of the pulmonary circulation, or postcapillary, with primary findings located within the pulmonary venous circulation, between the capillary bed and the left atrium. The arterial side has blood that has been oxygenated in the lungs and is being carried to the tissues of the body. The venous circulation has blood that has already passed through the capillaries and given up oxygen for the tissues and become charged with carbon dioxide and ready to pass through the respiratory organs to release its carbon dioxide and renew its oxygen supply.

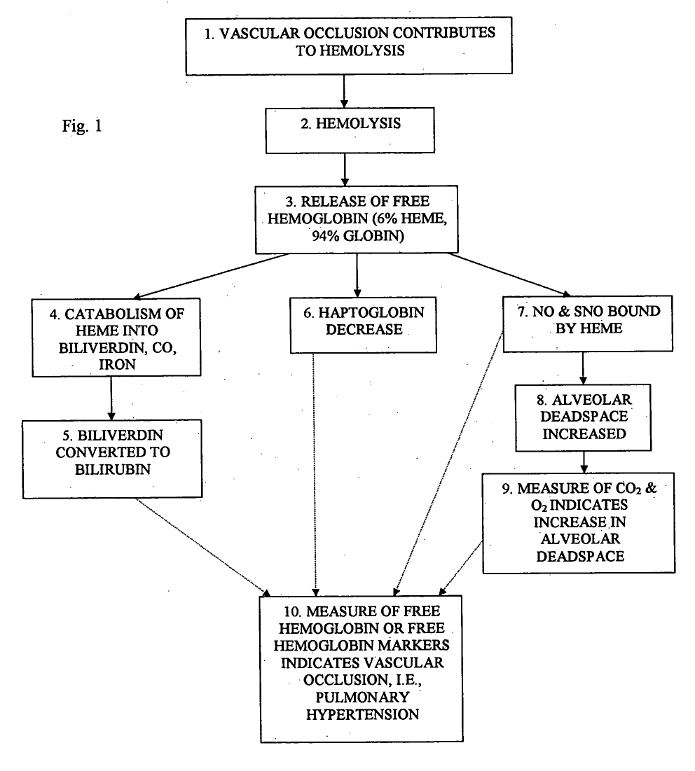

FIG. 1 is a diagram graphically representing a relationship between pulmonary hypertension and free hemoglobin and its surrogate markers. Precapillary pulmonary occlusion causes an increase in shear forces on red blood cells as they travel through the pulmonary capillary bed under higher than normal driving pressure, and is thus likely to cause turbulent or non-laminar blood flow. Precapillary pulmonary occlusion may cause tricuspid regurgitation, which is a disorder involving backward flow of blood across the tricuspid valve that separates the right ventricle (lower heart chamber) from the right atrium (upper heart chamber) and is a well-known consequence of pulmonary hypertension. This turbulence may cause shear forces to be imparted to red blood cells, leading to intracardiac hemolysis. Moreover, intrapulmonary vascular occlusion with associated pulmonary hypertension also contributes to increased intravascular shear forces and hemolysis in the lung vasculature (step 1). Accordingly, hemolysis can occur as a consequence of pulmonary hypertension. The term “hemolysis,” as used in the context of the present invention, refers to the destruction or dissolution of red blood cells (step 2), with release of hemoglobin as free hemoglobin (step 3). The term “hemoglobin,” as used in the context of the present invention, refers to the red respiratory protein of red blood cells that transports oxygen from the lungs to the tissues of the body and is composed of approximately 6% heme and 94% globin. Heme oxygenase enzyme that is present in various cells of the lung and other organs catabolizes heme to biliverdin, carbon monoxide (CO) and iron (step 4). The term “heme oxygenase enzyme,” in the context of the present invention, refers to an enzyme that is responsible for catabolism of the heme group. The term “catabolism,” in the context of the present invention, refers to the breakdown of complex substances into more simple ones with release of energy.

In a study to measure the effect of elevated pulmonary pressure in rats, which is discussed in greater detail hereinbelow, a 40-fold increase in expression of messenger RNA coding for the heme oxygenase enzyme was observed in rats with elevated pulmonary pressure. Messenger RNA mediates the transfer of genetic information from a cell nucleus to ribosomes in the cell cytoplasm, where it serves as a template for protein synthesis. Therefore, an increase in expression of messenger RNA coding for heme oxygenase enzyme indicates that more heme oxygenase enzyme was being synthesized to accommodate the increase in hemolysis in rats with elevated pulmonary pressure.

Once the heme group is broken down by the heme oxygenase enzyme, biliverdin is converted to bilirubin (step 5). Free hemoglobin, biliverdin, bilirubin and/or carbon monoxide (CO) may be detected in bodily fluids and/or expired breath samples (step 10). Accordingly, measuring the concentration of free hemoglobin, biliverdin, bilirubin and/or carbon monoxide in a patient's bodily fluids and/or exhaled breath may provide a way to indicate an elevated hemolysis rate and therefore, vascular occlusion and pulmonary hypertension.

It is well known that haptoglobin decreases as a consequence of the release of free hemoglobin with hemolysis (step 6). The term “haptoglobin,” as used in the context of the present invention, refers to a plasma protein that is a normal constituent of blood serum and functions to bind free hemoglobin, thereby retaining iron, in the bloodstream. Using the above-described model, it was discovered that the protein haptoglobin, and its precursor proteins, prehaptoglobin, were markedly decreased in the presence of the pulmonary vascular occlusions that caused the right ventricular pressure to exceed normal pressure. The haptoglobin concentration decreased in proportion to the magnitude of pulmonary hypertension in the dimensions of both time and pressure; i.e., higher pulmonary arterial pressures corresponded to lower haptoglobin levels. Further, elevated pulmonary arterial pressure for a longer period of time also appeared to correspond to lower haptoglobin levels; i.e., the longer the pulmonary arterial pressure was elevated, the more depressed haptoglobin levels appeared. Accordingly, measuring the concentration of a patient's haptoglobin may provide a way to indicate an elevated hemolysis rate and therefore, vascular occlusion and pulmonary hypertension (step 10).

An advantage of measuring haptoglobin in a patient's bodily fluid is that the concentration of haptoglobin, unlike the concentration of free hemoglobin, in sampled bodily fluid, specifically blood, is not spuriously elevated by sampling procedure. A common technical difficulty with drawing blood, especially from sick patients, which are prone to hypertension, is iatrogenic hemolysis. Iatrogenic hemolysis is hemolysis of the blood being sampled as it is aspirated into the blood tube due to the small diameter of the needle being used to draw the blood. Iatrogenic hemolysis causes the free hemoglobin to be spuriously elevated in the sample, therefore resulting in a false positive test. In contrast, haptoglobin concentration in the sampled blood does not go down from iatrogenic hemolysis.

It is well known that free hemoglobin binds nitric oxide. Further, nitric oxide (NO) and its thiol derivative (SNO) are well established mediators of pulmonary vascular dilation. As such, removal of NO or SNO from the pulmonary vascular bed may result in vasoconstriction. When hemolysis occurs, the heme portion of free hemoglobin binds NO and/or SNO (step 7), thereby causing vasoconstriction. Pulmonary vasoconstriction causes an increase in pulmonary vascular resistance, and as a consequence, causes an increase in pulmonary arterial resistance and pulmonary hypertension (step 10).

In theory, hemolysis can cause, perpetuate, and worsen pulmonary hypertension. Accordingly, it can be seen that pulmonary hypertension produces a vicious cycle of intracardiac and intrapulmonary hemolysis, followed by release of free hemoglobin, then reduction in NO and SNO, which causes worsened intrapulmonary vasospasm, which then worsens pulmonary hypertension. Pulmonary arterial vasoconstriction can, in turn, alter the ability of the lung to exchange carbon dioxide and oxygen.

Further, it is disclosed in an article entitled “Nitric oxide inhalation increases alveolar gas exchange by decreasing deadspace volume,” Crit. Care Med 29 (6):1195-1200, 2001), that the ability to measure the apparent alveolar deadspace volume can be a surrogate measurement to monitor the effect of nitric oxide therapy. More specifically, the article reports that inhaled nitric oxide causes a decrease in alveolar deadspace volume and alveolar partial pressure of carbon dioxide. As such, the article shows that alveolar deadspace volume is directly and linearly related to arterial-to-end tidal CO₂ partial pressure. These findings suggest that nitric oxide therapy can thereby increase alveolar gas exchange in injured lungs. Conversely, a measurement of the alveolar deadspace volume may be used as a means to monitor for the adverse effect of the withdrawal of nitric oxide from the lung vasculature, as in the case of increased free hemoglobin (step 8).

U.S. Pat. No. 6,575,918 to Kline, (the “'918 patent”), the entirety of which is herein incorporated by reference, discloses a way in which to measure alveolar deadspace volume. More particularly, the '918 patent discloses that the expired ratio of carbon dioxide (CO₂) to oxygen (O₂) and the plot of the CO₂ as a function of the O₂ can be used to detect the presence of pulmonary hypertension associated with increased alveolar deadspace (step 9). Higher levels of pulmonary hypertension correspond to higher apparent alveolar deadspace, manifested as a lower ratio of expired CO₂ to O₂ (step 10).

More specifically, pulmonary arterial thromboembolism, or severe vasoconstriction, impedes blood flow to alveoli, producing lung units with very high ratios of ventilation to perfusion. Thus, the experimental diagram of alveolar partial pressure of carbon dioxide (P_{CO2}), plotted as a function of alveolar partial pressure of oxygen (P_{O2}), predicts a low ratio of these gases (P_{CO2}/P_{O2}) in the alveolar compartment of patients with pulmonary vascular occlusion.

In an attempt to develop noninvasive methods to detect and monitor pulmonary hypertension, an experimental model of pulmonary hypertension has been employed. A detailed description of the model may be found in a previously published article entitled “Chemokines Accumulate in the Lungs of Rats with Severe Pulmonary Embolism Induced by Polystyrene Microspheres,” J. Immunol 171:5529-5536, 2003. Briefly summarized, in this model, plastic microspheres or beads of known diameter are infused into a rat's venous circulation. These beads are impervious and lodge into the rat's pulmonary arteries creating an occlusion. The term “occlusion” as used herein refers to an obstruction or closure of a passageway or vessel. By titrating the dose of the beads, the model can be used to produce pulmonary hypertension in the rats and to vary the severity and time course of development of pulmonary hypertension.

This experimental model has clinical relevance for humans because it produces precapillary occlusion and manifests gas exchange abnormalities, pleural effusion and pulmonary hypertension as is seen in humans with large pulmonary embolism. The term “pleural effusion,” as used in the present invention, refers to an exudation of fluid from the blood or lymph into a space located between the lung and the chest wall.

Generally, it is thought that as the severity of pulmonary embolism increases, the occurrence and severity of pulmonary hypertension also increases. Further, with pulmonary embolism, the mechanical vascular occlusion and the additional release of vasoconstrictive agents from the intrapulmonary thrombus, such agents including serotonin, prostanoids PGF2α, thromboxanes A and B, leukotrienes C4, D4 and E4, platelet activating factor and others, appear to produce a synergistic effect that causes acute pulmonary hypertension, worsened gas exchange and impaired right ventricular function.

Additionally, using the above-described experimental model, evidence of precapillary pulmonary vasoconstriction associated with pulmonary embolism has been shown, including visual evidence in the form of cineangiograms and physiological evidence in the form of gas exchange measurements, has been shown. The term “vasoconstriction,” as used in the context of the present invention, refers to constriction of a blood vessel.

In brief summary, it was shown that the induction of pulmonary embolism causes a reduction in the ratio of CO₂ to O₂ in expired breath and that such ratio increases with treatment effect, which is coincident with improved lung perfusion. In addition, direct pulmonary angiography was performed on the rats with cineangiographic analysis of pulmonary vascular perfusion to determine how the rats responded to treatment. The term “cineangiography,” as used in the present invention refers to the use of a movie camera to film the passage of a contrast medium through blood vessels for diagnostic purposes. A detailed discussion of these findings is discussed hereinbelow in Example 1 and is also available in an article entitled “Inhibition of prostaglandin synthesis during polystyrene microsphere-induced pulmonary embolism in the rat,” American Journal of Physiology—Lung Cellular & Molecular Physiology, 284:1072-1081, 2003, which is incorporated by reference herein.

Accordingly, a number of useful methods of detecting and monitoring for pulmonary hypertension and related phenomena are proposed. In most cases, the method begins with the collection of one or more bodily fluids from the mammalian subject. Examples of bodily fluids may include, but are limited to, whole blood, blood serum, plasma, urine, and expired breath condensate. In addition, the methods may begin with collection of expired breath in gaseous form. Methods and apparatuses suitable for collecting exhaled breath condensate have been described in several previous commonly-assigned patent applications, including provisional U.S. Patent Application Ser. No. 60/434,916 filed Dec. 20, 2002 and entitled “DISPOSABLE HAND-HELD DEVICE FOR COLLECTION OF EXHALED BREATH CONDENSATE FOR ASSAY OF BIOMARKERS FOR THE DETECTION AND PROGNOSIS OF LUNG ISCHEMIA,” and provisional U.S. Patent Application Ser. No. 60/447,581 filed Feb. 14, 2003 and entitled “DEVICE AND METHOD FOR COLLECTION OF EXHALED ALVEOLAR BREATH CONDENSATE,” the entirety of each of which is incorporated herein by reference.

Any of several markers for pulmonary hypertension, including free hemoglobin and its surrogates, and other phenomena may be measured in the collected bodily fluid or expired breath in order to effect diagnosis or the like.

For example, in a first preferred method of the present invention, the presence of acute or chronic pulmonary hypertension may be detected by measuring the concentration of haptoglobin, prehaptoglobin, free hemoglobin, and/or biliverdin and bilirubin in the collected bodily fluid. Similarly, the development of pulmonary hypertension in a mammalian subject may likewise be monitored by measuring the concentration of haptoglobin, prehaptoglobin, free hemoglobin, and/or biliverdin and bilirubin in the collected bodily fluid.

Monitoring a patient for the development of pulmonary hypertension may be particularly useful in the case of suspected venous thromboembolism, sickle cell disease, fat embolism syndrome, tumor embolism syndrome, amniotic fluid embolism, pulmonary schistosomiasis, congestive heart failure, chronic obstructive pulmonary disease, primary pulmonary hypertension, Eisenmenger's syndrome, canine dirofilariasis, and the like.

Once the presence of pulmonary hypertension has been detected, the response of a mammalian subject to treatment designed to reduce pulmonary arterial or vascular resistance may be assessed by collecting a bodily fluid and measuring the concentration of at least one of haptoglobin, prehaptoglobin, free hemoglobin, biliverdin, or bilirubin in the collected bodily fluid. Assessment may include assessing the response of the subject to inhalational drug therapy that is designed to reduce pulmonary arterial resistance and/or assessing the response of the subject to infused or ingested medications that are designed to reduce pulmonary vascular resistance.

Measurement of the various markers may be accomplished in a variety of ways, including the use of an immunoglobulin-based assay format, an enzyme-linked immunoassay, fluorescent methods, colorimetric assays, bioassays and/or radioisotopic techniques. A person of ordinary skill in the art would be familiar with each of these measurement methods.

In a second preferred method of the present invention, the presence of acute or chronic pulmonary hypertension may be detected by determining the ratio of the concentration of surrogate markers of free hemoglobin in expired breath. For example, pulmonary vasoconstriction due to intrapulmonary or free hemoglobin may be identified by determining the ratio of the concentration of expired carbon dioxide to expired oxygen.

The ratio of CO₂ to O₂ may be determined independently or by using any of a variety of other techniques, including, but not limited to, determining the ratio as a function of expired volume, plotting the dynamic expired carbon dioxide content as a function of dynamic expired breath oxygen content with each breath cycle, plotting any point estimate of carbon dioxide from any portion of the expired breath cycle as a function of the oxygen content measured simultaneously, plotting the mathematical equation describing the line that defines the ratio of expired carbon dioxide to expired oxygen as a function of expired volume with each breath, and/or determining the area under the curve of the plot of the ratio of expired carbon dioxide to expired oxygen as a function of expired breath volume.

It is a preferred aspect of this embodiment to determine the ratio of CO₂ to O₂ in expired breath and use such ratio in combination with a measured concentration value of one or more of haptoglobin, prehaptoglobin, free hemoglobin, biliverdin and/or bilirubin in one or more bodily fluids to determine whether a mammalian subject has pulmonary hypertension. As discussed above, the concentration of free hemoglobin and/or one or more of its surrogate markers may indicate hemolysis. Additionally, the ratio of CO₂ to O₂ may likewise indicate pulmonary hypertension. Using these two measured values in conjunction with one another provides a stronger link between hemolysis and pulmonary hypertension and provides stronger evidence of pulmonary hypertension.

The CO₂ to O₂ ratio may likewise also be determined in conjunction with measurement of the exhaled free gas concentrations or partial pressures of carbon monoxide and nitric oxide. One of ordinary skill in the art would understand and be able to perform the above-listed methods.

In addition to being used to detect the presence of pulmonary hypertension, surrogate markers of free hemoglobin in expired breath may also be used to evaluate the effect of treatments designed to reduce free hemoglobin. This evaluation is performed using the same test methods described above for determining the ratio of the concentration of expired carbon dioxide to expired oxygen in expired breath.

The method of the present invention may also be utilized to assess for intrapulmonary hemolysis and subsequent free hemoglobin release due to catheter or hydrojet fragmentation to treat intrapulmonary thrombosis. This may be accomplished by measuring the concentrations of expired nitric oxide and expired carbon monoxide or measuring the concentration of haptoglobin, prehaptoglobin, free hemoglobin, biliverdin, or bilirubin in one or more bodily fluids collected from a mammalian subject as described herein above.

It is also possible to identify the biological effect of free hemoglobin on the pulmonary vasculature by measuring the concentrations of expired gaseous nitric oxide and carbon monoxide or by measuring the concentrations of dissolved nitric oxide and carbon monoxide in expired breath condensate. Either of these measurements may be carried out as an independent measurement or may be carried out in conjunction with measurement of one or more of the above-described markers (haptoglobin, prehaptoglobin, free hemoglobin, biliverdin, or bilirubin) in a bodily fluid.

### EXAMPLES

### Example 1

The effect of pulmonary embolism on the ratio of CO₂ to O₂ in expired breath was measured in accordance with the present invention.

In order to create and control the condition of pulmonary hypertension, polystyrene microsphere beads with a mean diameter of 24 μm were infused into a rat's venous circulation at a rate of 0.1 ml/min to induce fixed pulmonary obstruction. These beads were impervious and lodged into the rat's pulmonary arteries creating an occlusion.

The concentration of expired CO₂ and O₂ in rats having induced pulmonary embolism was measured according to the following procedure. At 16 hours after pulmonary embolism induction, the rats were ventilated with a small-animal, mechanical ventilator (model 2094; Kent Scientific, Lithfield, Conn.). To measure ventilation parameters, a gas flow transducer (model TSD 137C; Biopac Systems, Santa Barbara, Calif.) was attached to the inspiratory limb of the ventilator circuit. End-tidal CO₂ was measured by a side stream quantitative CO₂ capnometer (model CO₂ 100A; Biopac Systems) attached to the expiratory limb. End-tidal O₂ was measured by a side stream paramagnetic oxygen sensor attached to the expiratory limb (model O200A; Biopac Systems). After instrumentation, all rats were given succinylcholine to relax breathing efforts, allowing end-tidal CO₂ measurements to be obtained from flat-topped, steady-state expirograms, during controlled, constant mechanical ventilation and without interference from interposed spontaneous breathing efforts.

End-tidal expired carbon dioxide (etCO₂), end-tidal expired oxygen (etO₂), partial pressure of carbon dioxide in arterial blood (P_{CO2}) and partial pressure of oxygen in arterial blood (P_{O2}) were all measured. Pulmonary gas exchange was measured primarily by the alveolar deadspace volume, which was calculated using etCO₂ in the Severinghaus equation. It is known that an increase in alveolar deadspace volume correlates with an increase in pulmonary vascular resistance.

Rats subjected to pulmonary embolism demonstrated a statistically significant decrease in peak partial pressure of carbon dioxide measured at end-tidal respiration and an increase in the nadir end-tidal partial pressure of oxygen measurement, which was coincident with increased alveolar deadspace volume and a significantly increased difference between the end-tidal partial pressure of oxygen and the arterial partial pressure of oxygen. Accordingly, as was hypothesized, in rats subjected to pulmonary embolism, the ratio of the partial pressure of carbon dioxide over the partial pressure of oxygen decreased in proportion to the alveolar deadspace volume increase.

### Example 2

The effect of pulmonary embolism on the concentration of haptoglobin in blood was measured in accordance with the present invention.

Blood specimens were obtained from patients with pulmonary embolism who underwent an echocardiography-Doppler study. Each patient's right ventricular pressure was estimated by incorporating the measured velocity of the “regurgitant jet” across the tricuspid valve and an assumed central venous pressure into a modification of the Bernoulli equation.

Each patient was categorized into one of three right ventricular pressure (RVSP) groups: Normal (no regurgitant jet or RVSP<20 mm Hg), Mild (RVSP 30-40 mm Hg), Moderate (RVSP 45-55 mm Hg) and High (RVSP>55 mm Hg).

Haptoglobin concentrations were measured using an ELISA assay, constructed with the use of a commercially available antibody against haptoglobin. One of ordinary skill in the art would understand how to perform the ELISA assay. For reference, haptoglobin was measured in the blood of healthy persons free of pulmonary embolism.

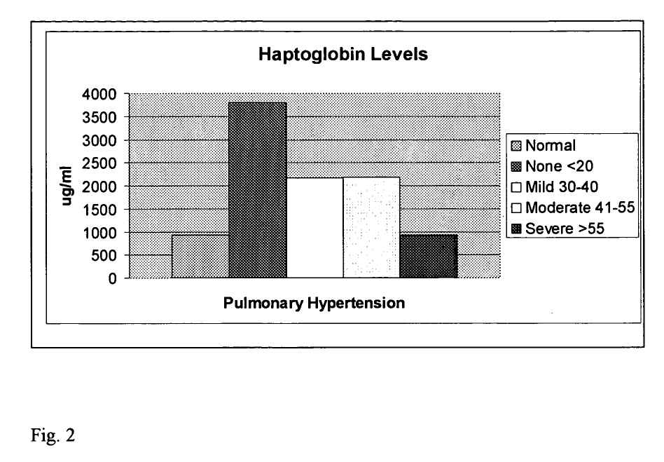

FIG. 2 is a graphical representation of the results of the ELISA assay analysis. The analysis indicated that patients having pulmonary embolism, but also having normal right ventricular pressure, had elevated haptoglobin concentrations relative to healthy patients (i.e., normal approximately 800-1200 ug/mL, versus >1500 ug/mL for patients with pulmonary embolism and normal pressures). This result suggests an “induction” effect that is consistent with the acute phase reactant nature of haptoglobin in response to the inflammatory effect of venous thrombosis.

However, as the right ventricular pressure increased, the haptoglobin concentration decreased compared to normal, and then serum haptoglobin concentrations decreased to subnormal concentrations (i.e., <800 ug/mL) in approximately 75% of patients with severe pulmonary hypertension. Accordingly, it can be seen that patients with pulmonary embolism with elevated haptoglobin concentrations are less likely to have associated pulmonary hypertension, whereas patients with pulmonary embolism and normal or decreased serum haptoglobin concentrations (i.e., below 1000 ug/mL) are likely to have associated pulmonary hypertension.

### Example 3

The effect of pulmonary embolism on the ratio of CO₂ to O₂ in expired breath was measured in accordance with the present invention.

A device as described in the '918 patent was used to measure the expired ratio of CO₂ to O₂ in 178 patients evaluated for possible pulmonary embolism. In addition, the same 178 patients underwent standardized evaluation techniques for pulmonary embolism as a means to verify results obtained using the breathing device.

The patients were asked to use a particular breathing technique when using the breathing device. Specifically, the patients breathed room air for at least two minutes. Then, while in semi-Fowler's position, and wearing nose clips, patients breathed into a duckbill-shaped mouthpiece in airtight connection with the airflow transducer. A research assistant provided help to the patient as needed. Patients delivered a sharp, rapid, deep exhalation to a maximum endpoint, starting from a midpoint of tidal breathing (i.e., not delivered after a sigh inspiration) followed by a few normal breaths, and then a 30 second period of tidal breathing. The term “tidal breathing,” as used in the present invention refers to a person's normal breathing while at rest and is in contrast to deep exhalation. This sequence was repeated twice more, yielding three deep exhalations and three 30-second samples of tidal breathing.

The same patients then underwent standardized evaluations for pulmonary embolism, including a D-dimer screening step in many cases, and then contrast-enhanced computerized tomography angiography of the chest and indirect venography, which was interpreted by board-certified body radiologists who were unaware of study results.

Table 1 shows the mean breathing device measurements from three deep exhalations for all 178 patients. P_{CO2} maximum is the maximum partial pressure of CO₂ in a given exhalation, P_{O2} minimum is the minimum partial pressure of O₂ in a given exhalation and P_{CO2} max/P_{O2} min is the ratio of the two previous numbers. PE+ indicates that the diagnosis was pulmonary hypertension and PE− indicates that the diagnosis was no pulmonary hypertension.

TABLE 1
Expired breath data obtained from three deep exhalations

| | | Deep exhalation | | |
|---|---|---|---|---|
| | | First | Second | Third |
| Parameter | Diagnosis | mean* | mean | mean |
| Breath volume (L) | PE+ | 1.13 | 1.14 | 1.13 |
| | PE− | 1.06 | 0.99 | 1.01 |
| P_{CO2} maximum | PE+ | 36.9 | 38.3 | 37.9 |
| | PE− | 45.3 | 45.2 | 44.5 |
| P_{O2} minimum | PE+ | 108 | 106 | 107 |
| | PE− | 98 | 99 | 100 |
| P_{CO2} max/P_{O2} min | PE+ | 0.36 | 0.38 | 0.37 |
| | PE− | 0.48 | 0.48 | 0.47 |

*Mean of one breath from 154 PE− patients and 24 PE+ patients

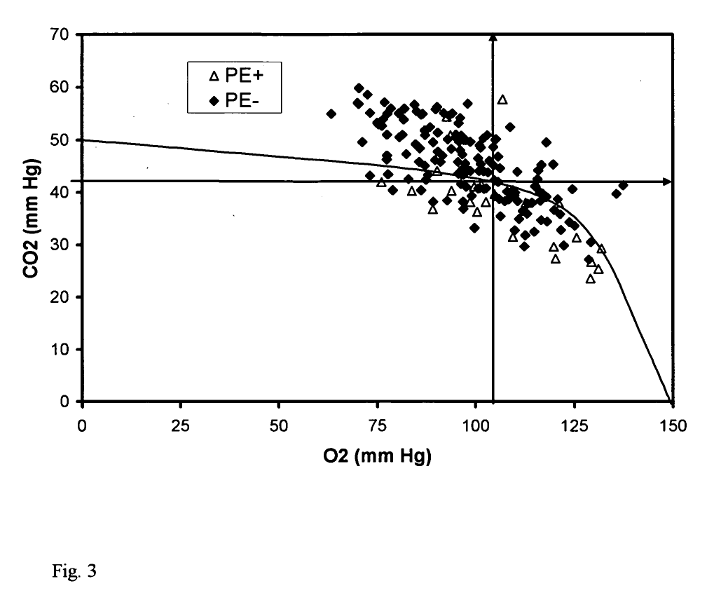

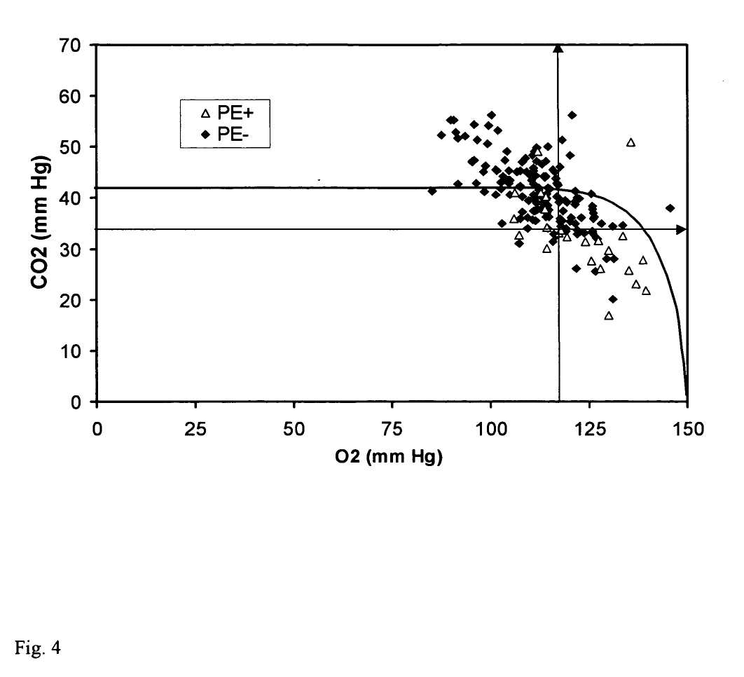

The diagnostic utility of expired P_{CO2} and P_{O2} was first investigated by plotting P_{CO2} versus P_{O2}. FIGS. 3 and 4 show the plot of P_{CO2} versus P_{O2} for 178 patients using the mean values of the first two deep exhaled breaths and the mean end-tidal P_{CO2} and P_{O2} obtained during the first minute of tidal breathing respectively. The dark curved lines represent the best-fit discriminate line that separates patients with pulmonary embolism (PE+) from those without pulmonary embolism (PE). Patients with PE+ tend to fall below the line, and patients without PE tend to be above the line. Each plot was divided into quadrants (straight lines with arrowheads) to separate out the patients based on probability as predicted by their location on the plot. Comparison of the lower right-hand quadrants suggests that the end-tidal P_{CO2} and P_{O2} data (FIG. 4) had a slight advantage over the deep-expired P_{CO2} and P_{O2} data (FIG. 3) as a rule in instrument.

Twenty-four (13%) of these 178 patients were ultimately diagnosed with pulmonary embolism. Some of these patients, both with pulmonary embolism and without pulmonary embolism, underwent echocardiography-Doppler examination, and were categorized as having no pulmonary hypertension versus moderate or severe pulmonary hypertension using the RVSP cutoff values described hereinabove in Example 2. It was found that patients with pulmonary embolism and moderate or severe pulmonary hypertension generally had a ratio of CO₂ to O₂ that was <0.30 whereas patients with moderate or severe pulmonary hypertension and no pulmonary embolism generally had ratio of CO₂ to O₂ of >0.30. The implication is that pulmonary hypertension as a consequence of pulmonary embolism causes more severe reduction in the ratio of CO₂ to O₂ than does pulmonary hypertension that is caused by other problems. When used in combination with pretest probability, the breathing device produced a relatively high specificity while retaining reasonable sensitivity.

Based on the foregoing information, it is readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention. Accordingly, while the present invention has been described herein in detail in relation to its preferred embodiment, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements; the present invention being limited only by the claims appended hereto and the equivalents thereof. Although specific terms are employed herein, they are used in a generic and descriptive sense only and not for the purpose of limitation.

1. A method of detecting and monitoring the presence of acute or chronic pulmonary hypertension in a mammalian subject, comprising:
(a) collecting a bodily fluid;
(b) measuring the concentration of free hemoglobin in the bodily fluid; and
(c) determining whether the concentration of free hemoglobin in the bodily fluid indicates the presence of pulmonary hypertension.

2. The method of claim 1, further comprising collecting expired breath, measuring the concentration of carbon dioxide and oxygen in the expired breath, calculating a ratio of the concentration of carbon dioxide to the concentration of oxygen in the expired breath and determining whether the ratio of carbon dioxide to oxygen in the expired breath in combination with the concentration of free hemoglobin in the first bodily fluid indicates the presence of pulmonary hypertension.

3. The method of claim 1, wherein the bodily fluid is a first bodily fluid, wherein the method further comprises collecting a second bodily fluid, measuring the concentration of dissolved nitric oxide and carbon monoxide in the second bodily fluid and determining whether the concentration of nitric oxide and carbon monoxide in the second bodily fluid in combination with the concentration of free hemoglobin in the first bodily fluid indicates the presence of pulmonary hypertension.

4. The method of claim 3, wherein the first bodily fluid is selected from a group consisting of whole blood, blood serum and plasma, and the second bodily fluid is expired breath condensate.

5. A method of detecting and monitoring the presence of acute or chronic pulmonary hypertension in a mammalian subject, comprising:
(a) collecting a bodily fluid;
(b) measuring the concentration of an at least one surrogate marker of free hemoglobin in the bodily fluid; and
(c) determining whether the concentration of the surrogate marker of free hemoglobin in the bodily fluid indicates the presence of pulmonary hypertension.

6. The method of claim 5, further comprising collecting expired breath, measuring the concentration of carbon dioxide and oxygen in the expired breath, calculating a ratio of the concentration of carbon dioxide to the concentration of oxygen in the expired breath and determining whether the ratio of carbon dioxide to oxygen in the expired breath in combination with the concentration of an at least one surrogate marker of free hemoglobin in the bodily fluid indicates the presence of pulmonary hypertension.

7. The method of claim 5, wherein the at least one surrogate marker of free hemoglobin in the bodily fluid includes haptoglobin.

8. The method of claim 7, wherein the bodily fluid is a first bodily fluid, wherein the method further comprises collecting a second bodily fluid, measuring the concentration of dissolved nitric oxide and carbon monoxide in the second bodily fluid and determining whether the concentration of dissolved nitric oxide and carbon monoxide in the second bodily fluid in combination with the concentration of haptoglobin in the first bodily fluid indicates the presence of pulmonary hypertension.

9. The method of claim 8, wherein the first bodily fluid is selected from a group consisting of whole blood, blood serum and plasma and the second bodily fluid is expired breath condensate.

10. The method of claim 7, wherein a haptoglobin concentration of less than or equal to 1000 ug/mL indicates the presence of pulmonary hypertension.

11. The method of claim 5, wherein the at least one surrogate marker of free hemoglobin in the bodily fluid includes prehaptoglobin.

12. The method of claim 5, wherein the at least one surrogate marker of free hemoglobin in the bodily fluid includes biliverdin.

13. The method of claim 5, wherein the at least one surrogate marker of free hemoglobin in the bodily fluid includes bilirubin.

14. The method of claim 5, wherein the at least one surrogate marker of free hemoglobin in the bodily fluid includes dissolved nitric oxide and carbon monoxide.

15. A method of detecting and monitoring the presence of acute or chronic pulmonary hypertension in a mammalian subject, comprising:
(a) collecting expired breath;
(b) measuring the concentration of an at least one surrogate marker of free hemoglobin in the expired breath; and
(c) determining whether the concentration indicates the presence of pulmonary hypertension.

16. The method of claim 15, wherein the at least one surrogate marker of free hemoglobin includes carbon dioxide and oxygen.

17. The method of claim 16, further comprising determining a ratio of the concentration of carbon dioxide to the concentration of oxygen.

18. The method of claim 17, wherein a ratio of carbon dioxide to oxygen of less than 0.30 indicates the presence of pulmonary hypertension.

19. The method of claim 15, wherein the at least one surrogate marker of free hemoglobin includes expired nitric oxide and carbon monoxide content.
